(19) United States
(12) Patent Application Publication
(10) Pub. No.: US 2019/0298767 A1
(43) Pub. Date: Oct. 3, 2019

(54) Nucleic acid molecule and method to make biallelic modifications in a target gene or locus which is part of the genetic material of a cell

(71) Applicants: Yacob Gomez Llorente, New York, NY (US); Victor Galvez Jerez, Badajoz (ES)

(72) Inventors: Yacob Gomez Llorente, New York, NY (US); Victor Galvez Jerez, Badajoz (ES)

(21) Appl. No.: 16/139,021

(22) Filed: Sep. 22, 2018

(30) Foreign Application Priority Data
Mar. 28, 2018 (ES) 2634802

(51) Int. Cl. A61K 35/17; A61P 31/18; C12N 5/00; C07H 21/02; C12N 15/10; A61K 48/00; C12N 15/65; C12N 15/63; C12N 15/113

(52) U.S. Cl. CPC A61K 35/17; A61P 31/18; C12N 5/00; C07H 21/02; C12N 15/1135; A61K 48/005; C12N 15/65; C12N 15/635; C12N 15/1131; C12N 15/102

(57) ABSTRACT

The aim of this invention is to provide a nucleic acid molecule and a method to modify at the same time both alleles of a target gene or region of the genome of a cell. This nucleic acid molecule has the ability to edit and modify both alleles of a gene or locus, currently present in the genetic material of a cell. The nucleic acid molecule encodes for certain nuclease proteins which once expressed will cleave the target gene or locus in the cell DNA. Afterwards the nucleic acid molecule will integrate itself in the cleavage site in at least one of the two alleles at first, by means of the innate homologous recombination repair mechanism of the cell. That is possible due to the homology regions that the introduced molecule is carrying, homologous to the target gene or locus. Once the nucleic acid molecule is integrated in the first allele, the nucleases will eventually produce another cut in the remaining allele and, by using again the homologous recombination repair pathway of the cell, the cleaved allele will be repaired using as a template the previously modified allele, producing the integration of the molecule in the second allele of the target gene or locus. After the nucleic acid molecule has been integrated in both alleles, the activation of the transposable element encoded in the molecule will remove all the undesired sequences leaving only the desired modifications in the target gene or locus. Such modifications will be present and will be identical in both alleles making possible by this method the generation of mutations in wild type genes, the insertion of complete genes in genomes, the insertion and removal of specific sequences and the repair of genetic mutations present in the genome, among other uses.

### FIELD OF APPLICATION

The invention described in this patent has applications in the biotechnology and biomedical fields. This invention precisely belongs to the sector of gene therapy and gene editing.

The contents of this patent describe both the design of a nucleic acid molecule and a method to modify the genetic material present in a cell by using that molecule. This modification in the cell is permanent and has homozygous character, being present in both alleles for the selected gene or locus. The system is very versatile, being possible to edit the genome of any organism by using a protocol that requires a short period of time.

### STATE OF THE ART

### Introduction and Background

Any gene editing method based on homologous recombination requires to use at least two DNA vectors. Both of them have to be transfected to the cell at the same time.

The first one will code a molecular tool which, after expression, will make a double cut in the target gene. We will refer to this molecular tool as “nuclease” from now on.

The second vector would consist in a DNA sequence that carries the modifications to be introduced in the target gene. Flanking that area, the vector would also have homologous regions to the target gene. We will refer to this vector as “homologous recombination vector” from now on.

After the incision with the nuclease in the target gene, the cellular machinery may try to repair the cut by using their innate repair mechanism by homologous recombination and using the introduced homologous recombination vector as a template. As a consequence, the modifications carried in the template will be introduced in the cellular DNA.

This method, when useful to modify one of the two alleles, has proven to be inefficient to modify both alleles at the same time for several reasons:
i) the simultaneous co-transfection of two or more vectors is required.
ii) there are other innate DNA reparation systems available in the cell which can act originating a different DNA alteration than the desired one. These are: the NHEJ^{[1]} pathway (non-homologous end joining pathway) by which the two opened DNA terms join again generating random mutations; and the homologous recombination pathway by which the other unaltered allele of the gene is used as a template to repair the altered allele.
iii) the expression of the nucleases is episomal and therefore transitory.
iv) the probability of occurring two successful reactions of homologous recombination simultaneously in the same cell, one per allele, is extremely low. That is due to the reduced efficiency and therefore probability of every step of the reaction. Two simultaneous reactions require a double number of consecutive steps. Being each step probabilistically unlikely, the possibilities of occurring two consecutive events of homologous recombination in the same cell decrease geometrically. To achieve it, a high amount of multiple transfections in a high amount of cells is required for a single successful event.

All these reasons make basically impossible to select a cell or clone with both alleles of the genome homozygously modified (carrying the same modified sequence in both alleles). Usually two sequential experiments of gene editing are necessary, once some cells are selected carrying a single modified allele. As consequence there is an excessive in-vitro manipulation and often each allele carries different modifications. Additionally, there is not an existent detection mechanism to differentiate cells carrying a single modification, in just one allele, from the cells in which both alleles have been modified.

The invention exposed in this patent not only bypasses the current difficulties but also:
i) increases the efficiency of the gene editing process.
ii) uses the cell repair mechanisms to its own advantage to edit both alleles homozygously.
iii) makes possible the detection, isolation and expansion of the modified cells, allowing an exhaustive characterization. This is guarantee of a genetic stability analog to the native cells, making them suitable to be used for gene therapy.

### EXPLANATION OF THE INVENTION

The aim of this invention is to provide a nucleic acid molecule and a method to modify the two alleles of a cell in a target gene or a specific region of the genome. These previous described concepts namely: “nucleic acid molecule”, the related method and the target gene, locus or specific region of the cellular genome, will be in the context of this patent henceforth referred to as: “the molecule” or “the nucleic acid molecule”, “the method” and the “target DNA” or “target material”, respectively.

The first aspect of the invention refers to a molecule consisting in several regions as follows: Two homologous regions to the target gene or locus in the genetic material to edit (H regions). These will flank two regions which define a transposable element (T regions) which finally flank a single region which codes a group of proteins necessary for the method to work (R region) (FIG. 1).

Additionally the nucleic acid molecule carries one or more modifications to be permanently incorporated in the target gene (E region) which will be present in the H region as at least a single punctual modification of the target sequence, or inserted between the H and T regions and/or T and H regions encoding, e.g. and without limitation, a sequence, part of a gene or the whole gene, an intron or an exon.

Brief explanation of the mechanism of biallelic gene edition after the introduction of the nucleic acid molecule inside the cell:

The R region existing in the nucleic acid molecule encodes several nucleases, among others, as described in the section “Claims”. These nucleases, once they are expressed, recognize and cut the target gene or locus in the genetic material of the cell within a specific sequence. The cut occurs in at least one of the two existing alleles of the gene or locus.

Both H regions in the molecule have a high degree of homology with the regions surrounding the cleavage site in the cellular allele. These H regions are recognized by the cellular homologous recombination repair mechanism and they are used as template to repair the cut, introducing in the process the whole molecule of nucleic acid in the repaired allele.

After the molecule is integrated in one of the alleles, the expression of the genes contained in the R region becomes stable and therefore the expression of the nucleases, which cut again the other allele for the target gene or locus.

This allele is in turn repaired by the cell DNA repair system by means of homologous recombination using as template the complementary allele, already modified. After that happens, the genetic modification becomes therefore homozygous (the modified character is present in the two alleles) while before this step it was only heterozygous.

After the nucleic acid molecule which carries the modification has been inserted in the target alleles inside the cellular DNA, the transposable element coded in the T regions is activated. This activation removes all the unwanted sequences inserted by the integration of the molecule from the cellular DNA (R region and the two T regions). The desired modifications are the only ones that remain in the DNA of the cell. These modifications will be permanent and identical in both alleles.

The second aspect of the invention refers to the method to modify the genetic material of the cell in a way in which the modification is present in both alleles for the target gene, target locus or target region in the genetic material. This method includes the following steps:
i) Provide the nucleic acid molecule and introduce it in the cell.
ii) Select the cells which have integrated the nucleic acid molecule in both alleles for the target DNA.
iii) Trigger the excision of part of the introduced nucleic acid molecule in a way in which only the desired modifications (such as substitutions, deletions or additions of material in the sequence) remain in both alleles of the cell genetic material.
iv) Select the cells which present the desired modifications in both alleles.

The nucleic acid molecule subject of this invention and the designed method may be useful for the treatment of hereditary diseases either recessive or dominant, because the method makes possible the gene editing of both alleles at the same time. Therefore, another aspect of the invention is referred to the nucleic acid molecule subject of the invention as a therapeutic compound and its usage as a medicine.

Another aspect of the invention consists in the treatment and/or prevention of the acquired immunodeficiency syndrome resulting of either the HIV virus infection or related to a monogenic disorder. That aspect comprehends the administration of an effective therapeutic amount of the nucleic acid molecule to the organism or affected subject and/or the re-administration of cells from the same subject once they have been previously modified by the proposed method. The organism or subject may also be human.

### DETAILED DESCRIPTION OF THE INVENTION

The first aspect of the invention is referred to a nucleic acid molecule. This molecule is composed by the following regions in the transcription direction 5′ to 3′:
H: first region of homology to the target DNA which is intended to be edited.
T: region which encodes the beginning of a transposable element.
R: region which encodes a group of proteins necessary for the method to work.
T: region which encodes the end of a transposable element.
H: second region of homology to the target material which is intended to be edited.

According to the transcription direction 5′ to 3′, the nucleic acid molecule will show the basic structure H-T-R-T-H.

The regions of homology (henceforth referred to as “H regions”) existing in the nucleic acid molecule will define the homology branches (5′ homology branch and 3′ homology branch in the transcription direction 5′ to 3′). These H regions are necessary for the nucleic acid molecule to be used as template of the homologous recombination based DNA repair system. Therefore, they are necessary to carry out the integration of this molecule in the cell genetic material. The H regions are analogous to the regions in the cellular genome located before and after the nuclease recognition site (defined afterwards in this document).

By homology region is defined a sequence of DNA with high identity (high percentage of analogy) to a target sequence. This target sequence is a DNA sequence existing the gene or locus target of modification. By means of the cellular process of homologous recombination, such sequences may be recognized and exchanged.

The H regions in the nucleic acid molecule may be of variable length, from 100 bp to 3 kb long. For a preferred embodiment their length will be between 900 bp and 1 kb.

### PARTICULAR EMBODIMENTS

In a particular embodiment, the H regions in the molecule don't contain any modifications (mutations) with regard to the target DNA (FIG. 2a).
In another particular embodiment at least one of the H regions in the molecule contains at least one modification (mutation) with regard to the target material. If there are several mutations, this method will refer to them as E region (E from “editing”). This E region may have certain degree of homology to the target material and will be present within the H regions.
During the gene edition process this modifications will become a constitutive part of the target DNA (FIG. 2b).
In another particular embodiment the molecule consists in one or more E regions which contain the genetic modifications to incorporate to the target material. During the gene edition process these modifications will become part of the target gene in the cellular genetic material.
In another particular embodiment, the E region of the nucleic acid molecule won't have any homology with the target gene or locus to edit and will be interspersed between the H and T regions of the molecule. This E region may encode for example and not limited to: a sequence, part of a gene, an intron, an exon or a whole gene. During the gene editing process the E region will become a constitutive part of the target material, being inserted in a specific point of the target gene or locus in which it was not before. The insertion point of the E region in the cellular genome will be determined by the sequence of the H regions. Based on that, the E region may be placed with exact accuracy in the target gene or locus present in the cellular genetic material, being therefore possible for example and not limited to add or subtract sequences without altering the reading frame of the target gene.
In another particular embodiment, the nucleic molecule contains a E region placed between the H and T regions following the transcription direction 5′ to 3′. In another particular embodiment, the E region is placed between the T and H regions following the transcription direction 5′ to 3′. In a third particular embodiment, there would be two E regions containing the genomic modification located between both H-T and T-H regions, following the transcription direction 5′ to 3′ (FIG. 2c).
In another more particular embodiment the nucleic acid molecule may contain one or more modifications in the sequence of at least one of the homology H regions and/or contain at least one E region (FIG. 2d).

The previously mentioned modifications may have the purpose of editing the genetic information of the target gene or locus to correct a present mutation, to generate a mutation, or to insert part of a gene, a whole gene or a sequence in the target locus. Such modifications may include the use of modified bases or nucleotides or known analogs to the natural nucleotides.

These modifications may have the purpose of modifying the target gene without changing what it encodes. Also these modifications may be silent mutations which don't alter the code of the target gene or locus. The purpose of these modifications may for example and not limited to be altering the sequence to create or disrupt: target sequences of restriction enzymes and/or nuclease recognition sites and/or primer binding sites and/or enhancers (activator protein binding sites) and/or inhibitor protein binding sites and/or binding sequences of other enzymes.

The method and molecule may also be used to remove part of the sequence of the target DNA (FIG. 3). For this purpose in a particular embodiment the H regions in the molecule are homologous to the sequence of the target gene or locus to be preserved, but they are designed to leave a separation of equivalent length to the sequence to be removed, i.e.: their sequences don't overlap with the target gene sequence, leaving a separation between them. Such separation corresponds to the fragment of the target gene which is removed from the genetic material of the cell after the gene edition is complete. The method for this particular embodiment is the same as the previously explained. The nucleases encoded in the molecule, once they are expressed, recognize and cut one allele of the target gene (FIG. 3a) and by means of the innate cellular repair mechanism of homologous recombination, the molecule is integrated in the cleaved allele (FIG. 3b). The nucleases cut the other allele of the target gene and the cut is repaired by homologous recombination using the previously modified allele as template. After both alleles are modified the transposon, composed by the regions TRT in both alleles, is splitted leaving as a result an homozygous deletion mutation in the sequence of the target gene (FIG. 3d).

The nucleic acid molecule subject of this invention consists in two T-regions which codifies for the beginning and end of a transposable element. These transposable elements may be but they are not limited to: LoxP sequences, including all the possible variations; FRT sequences, including all the possible variations; piggyBac transposon coding sequences and related sequences (ITR), including all possible variations; Sleeping Beauty coding sequences and related sequences (IR/DR), including all possible variations; and also the coding sequences for the Sandwich transposon and related sequences (IR/DR), including all possible variations; and other possible transposable sequences.

In a particular embodiment for the nucleic acid molecule the encoding sequences IR/DR of the Sleeping Beauty transposon are used as T-regions, and in a preferred embodiment the encoding sequences ITR of the piggyBac transposon are used as T-regions.

In the event of editing a gene with the system proposed by this patent it is necessary to take into account the residual sequence which the transposable elements leave after the excision from the genome. In case of the piggyBac transposon, it will leave additional nucleotides after the excision, consisting in the sequence TTAA. In the case of the Sleeping Beauty transposon the excision leaves additional nucleotides consisting in the sequence TA. In the case of the excision of two sequences from the LoxP recombination, their excision will leave a LoxP sequence integrated in the genome and another one in the splitted episomal element. In the case of the excision of two FRT recombination sequences, their excision leaves a FRT sequence integrated in the genome and another one in the splitted episomal element. In order to achieve a seamless edition, we recommend that the transposon encoded in the recombination template replace a native TTAA or TA site (if piggyBac or sleeping beauty transposons are used). In such a way, once the transposon is excised the missing TTAA or TA site is regenerated.

The nucleic acid molecule subject of this invention contains the R-region between two T-regions. In the context of this invention the transposable element consists in the sequence TRT which will be cleaved from the genome once the integration of the nucleic acid molecule has been occurred in the two alleles of the target gene or locus.

It is understood as “transposable element” in the context of this patent a region included inside the nucleic acid molecule subject of the invention defined by a region which encodes the beginning of the transposable element (T-region), followed by a region which encodes a set of proteins necessary for the realization of the invention (R-region) and followed by a region which encodes the end of the transposable element (T-region).

The scission of the TRT transposable element depends on the activity of a recombinase protein such as by way of example and without limitation, the recombinase Cre and all its possible variations, the recombinase Flipase and all its possible variations, the transposase SB-transposase and all its possible variations (such as SB10X and SB100X), the transposase PB-transposase and all its possible variations (such as HyPBase and ePBase). The scission removes the transposable element contained in the nucleic acid molecule removing all the integrated T and R-regions from both alleles of the genetic material, leaving the desired mutations and/or E-regions accurately placed in both alleles.

In a particular embodiment of the method proposed in this patent, the expression of the recombinase may be obtained by inserting a nucleic acid molecule in the cells which encodes such recombinase under the control of a promoter.

In another particular embodiment of the nucleic acid molecule, the coding of the recombinase which is needed to cleave the transposable element may be included in the R-region between the two T-regions which define the transposable element. The expression and/or function of the recombinase may be activated for example and without limitation, by mechanisms of controlled expression by an inducible or a repressible promoter, activator or repressor molecules, translation repression by interference RNA, etc.

The R-region carries sequences which encodes several proteins needed for the method to work properly. There are essential sequences in the R-region for the method like the N-sequences which encode for the nucleases and the S-sequences which encode for the selection genes. The R-region could contain other optional sequences, not needed for the method to work, although they can make easier the targeting, selection and expansion of the selected clones like the M-sequences which encode for marker proteins and the P-sequences which encode for proteins of cell proliferation.

The organization and the order of the R-region sequences N, S, M and P in the transcription direction 5′ to 3′ is irrelevant on the condition that their expression is guaranteed. Therefore, these sequences can be organized as polycistronic genes, as singular genes or as a combination of both types. Considering the direction 5′ to 3′ of the transcription for these sequences, they can be sorted in any order or permutation on the condition that they are flanked by the sequences which we are defined as T-regions. The reason to this is, as we described before, that the R-region is part of the transposable element and will be cleaved from the target gene or locus in both alleles (FIG. 4).

In a preferred embodiment the R-region present in the nucleic acid molecule, will be composed in the 5′ to 3′ direction of transcription, by a polycistronic gene which carries the N-M sequences followed by a second gene which encodes for the S-sequence.

Below the characteristics of the sequences N, M, S and P are highlighted:
The N-sequence existing in the R-region of the molecule encodes the nuclease proteins. These nucleases are necessary to carry out the method because they execute the cut in the two alleles of the target DNA. This sequence encodes for nucleases like by way of example and not limited to: homing endonucleases (HEs) and their variants, zinc finger nucleases (ZFN) and their variants, TALEN nucleases (from transcription activator-like effector nucleases) and their variants or RNA-dependent DNA endonucleases from the CRISP/Cas9 system (from clustered regulatory interspaced short palindromic repeats) as well as the related gRNA (guide RNA) and their variants (like CRISPR-Cas9 nickase). These nucleases may be of the previously described types but they are not limited to them.
   It is essential for a proper performance of the method that the nucleases are designed to recognize and cut the target DNA in the homology area delimited by the two H-regions, so that the regions of the target gene or locus contiguous to the cutting site show homology with the H-regions of the nucleic acid molecule.
The S-sequence present in the R-region of the molecule encodes the proteins of resistance and selection. The S-sequence is necessary to carry out the method because it allows the selection of:
   i) Cells or clones which have undergone at least one cutting event and at least one event of integration of the nucleic acid molecule in at least one of the two alleles of the target gene or locus.
   ii) Cells or clones which have undergone the scission event of the TRT transposable element present in the nucleic acid molecule integrated in the two alleles of the target gene or locus.
The S-sequence encodes resistance and selection proteins both for negative and positive selection and/or proteins with double selection function, positive and negative at the same time. In the context of this invention it is understood by “positive selection” the resistance to antibiotics or any other toxic molecule granted to the cell through the expression of a protein. By “negative selection” is understood the lysis effect produced in the cells as a consequence of the expression of a protein or its metabolic activity on a innocuous precursor that generates a toxic product.
The S-sequence may encode by way of example and without limitation, at least a protein which confers resistance to antibiotics or lytic proteins (positive selection).
The S-sequence may encode by way of example and without limitation, at least a protein which allows negative selection of the cells or clones like:
   The protein encoded by the thymidine-kinase gene of herpesvirus (HSV-TK) which, in the presence of ganciclovir or fialuridine, kills the cell.
   The protein encoded by the gene iCasp-9 which kills the cell in the presence of a dimerizing agent (AP20187 or B/B-Homodimerizer) which induces protein dimerization and subsequent activation.
   Proteins encoded by genes which expression induce lysis so that they are activated following the stimulation of inducible promoters (like the systems activated by tetracycline Tet-On, Tet-OFF and pTRE3G) or they stopped being repressed if their expression is controlled by a repressor system.
   In a particular embodiment the S-sequence, present in the nucleic acid molecule, encodes at least one bi-functional fusion protein like for example the protein derived from the gene puDeltatk, which confers puromycin resistance and induces cell lysis in the presence of ganciclovir or fialuridine.
Additionally, the R-region of the nucleic acid molecule subject of this invention may contain M-sequences encoding for marking proteins. The M-sequence is not essential for the method to work, but facilitates to a great extent the tasks of identification and selection of the cells which have integrated such nucleic acid in the target DNA. This selection may be carried on by means of many different techniques depending on the kind of marking protein (M) that is encoded in the molecule.
   In a preferred embodiment the M-sequence encodes at least one fluorescent marking protein like, as a way of example and without limitation: green fluorescent protein (GFP), Turbo GFP, copGFP, tdTomato, infrared fluorescent protein (IRFP), mEmerald, Venus, super yellow fluorescent protein 2 (SYFP2), DsRed, enhanced blue fluorescent protein (EBFP), enhanced yellow fluorescent protein (EYFP), Cerulean or enhanced cyan fluorescent protein (ECFP). The fluorescence emitted by the protein is used to identify and select the cells by means of flow cytometry, so that we will select only the cells which have a stable expression of the genes present in the R-region of the nucleic acid molecule.
   In a particular embodiment the M-sequence encodes at least one cell surface marker protein. We understand as “cell surface marker protein” in the context of this invention any protein localized in the surface of the cell which may be used to detect and isolate the cells that express that protein from the cells that don't do it by using techniques like, as a way of example and without limitation: flow cytometry, cell magnetic isolation and separation, antibody detection, immunopanning, etc. Some examples of cell surface marker proteins are, without limitation: leukocyte differentiation markers and clusters of differentiation (CD).
Additionally, the R-region of the nucleic acid molecule subject of this invention may contain P-sequences encoding for cell proliferation proteins. In the context of this invention, we understand as “cell proliferation protein” any protein expressed inside the cell which stimulates proliferation and division and/or inhibits apoptotic pathways. Some example of proliferation proteins are, without limitation: inhibitor of apoptosis proteins (IAP's), caspase activation pathway inhibitors (crmA, p35, Bcl-2, etc.) and immortalization proteins (EBNA-LP, hTERT, H2RSP, etc.).
   Additionally, the R-region of the nucleic acid molecule subject of this invention may contain P-sequences encoding for one or more interfering RNA related to cell proliferation events. In the context of this patent, we understand as “interfering RNA” any of the RNA molecules which regulates the expression pattern of the genes in the cell genetic material. These molecules comprise three large groups of RNA molecules known as small interfering RNA (siRNA), microRNA (miRNA) and PIWI-interacting RNA (piRNA). Examples of interfering RNA related to cell proliferation include, without limitation: cyclin-dependent kinase inhibitors (miR-24) and immortalizing miRNAs (miR-155).

Another aspect of the invention is referred to the method to modify the genetic material of the cell in a way in which the resulting modification or gene editing is produced in both alleles of the target gene or locus. This method comprises the following steps:
i) Provide and introduce in the cell the previously mentioned nucleic acid molecule in a way in which it can reach the cell nucleus and express the genes located in the R-region. At that moment the introduced molecule has a episomal character and therefore the expression of all the genes in the R-region (N, S, M and P-sequences) is transitory unless the following events occur: 1) the nuclease designed for cleaving the GOI and encoded by the N-sequence of the molecule recognizes the cleavage sequence in the target gene or locus of the cell genetic material and executes the cut (FIG. 5a). 2) A homologous recombination repair event is produced between regions of the genome flanking the target site and the two H-regions in the molecule which has been introduced in the cell (FIG. 5b). According to these two events, the molecule has had to be integrated in one of the two alleles of the target gene or locus present in the cell. Once it is introduced, the expression of the genes in the R-region (N, S, M and P-sequences) become stable. At that moment the allele that carries the integrated molecule will behave as a homing endonuclease gene^{[2]}, and the constitutive expression of the nucleases (encoded by the N-sequences) will produce the cut in the target sequence in the other allele (FIG. 5c). If the cut is repaired by the cell repair mechanism NHEJ, the target sequence will be regenerated after the cut, and the nucleases will cut it again until a repair event by homologous recombination occurs, using as template the allele which carries the molecule, copying it where the cut has been produced. At that moment, both alleles have integrated the molecule in homozygosis duplicating the set of genes in the R-region present in the cell genome (FIG. 5d).
ii) Select the cells which have integrated the molecule inside both alleles of the target gene or locus. Such selection is performed by means of the expression of the S-sequence by the cell to acquire resistance to antibiotics or other toxic compounds (positive selection). The selection is favored to a great extent by the expression of the M-sequence due to the possibility of detecting marker proteins by flow cytometry and other techniques, which make possible to detect the rise in the gene pool and therefore the expression in the cells modified in the two alleles. This process allows to select all the cells in which the integration of the molecule has been produced in the target gene or locus.
iii) Trigger the scission of the proper part of the molecule to keep only the desired modification in both alleles of the genetic material of the modified cells. In order to achieve that, the transposable element, delimited by the TRT regions, is activated by the different mechanisms explained before, depending on the different nature of the transposable element. The TRT transposable element, after the excision, becomes episomal and it is degraded in a short period of time by the cell machinery (FIG. 5e). This degradation makes all the genes encoded by the R-region disappear, and therefore the function associated to their expression.
iv) Select the cells which show the desired modifications in both alleles and which have removed the TRT transposable element: this selection is carried out thanks to the selection genes (S) present in the R-region by a negative selection event. The cell that keeps the TRT element either integrated, randomly reintegrated or episomal, will die. After the selection process the remaining cells will have only the desired genetic modifications integrated in both alleles.

In the context of this invention, the terms “nucleic acid”, “sequence” and “base pairs (bp)” are referred respectively to “desoxyribonucleic acid” (or also “ribonucleic acid”), “nucleotide sequence” and the length of the sequence based on the number of nucleotides that the sequence contains. Also, in the context of this invention, the term “nucleic acid” defines linear molecules as well as circular molecules, either single or double stranded. These terms may also include synthetic nucleotides analogue to natural nucleotides, as well as modified nucleotides but respecting the pairing code with the original nucleotides.

When the nucleic acid molecule subject of the invention is a desoxyribonucleic acid, its sequence will be adapted to the preferred codon usage for that specific organism, either animal, plant or human.

The introduction of the molecule in the cell or in the subject may be carried out as a way of example and without limitation, by means of either viral vectors or other vectors which contain such molecule or nonviral physicochemical methods known by the specialist scientist.

In a particular embodiment, the molecule is transfected as a plasmid. In another particular embodiment, it is transfected as a minicircle through a nucleofection method. In another particular embodiment, the molecule is transduced by means of viral vectors.

In a particular embodiment the molecule is administered to a cell culture (in vitro) and, once the method has been successfully ended, the cells are introduced in the organism or patient (ex vivo). In another particular embodiment the molecule is administered directly to the organism or patient (in vivo).

In the context of this patent by “recognition sequence”, “recognition site” and “binding site”, are understood specific sequences in the genetic material of the cell which is recognized and bound by a protein or polypeptide such as way of example and without limitation, the nucleases and other restriction enzymes. The cut may be produced within the sequence or but also in the surrounding area.

In the context of this patent the cell may be of a human, plant or animal origin. In a preferred embodiment the cells are of human origin and are, as a way of example and without limitation: hematopoietic stem cells (HSC), extracted from bone marrow through biopsy or from blood units of umbilical cord, lymphocytes or induced pluripotent stem cells (iPS), or other cells from the patient or organism extracted through biopsies or from explants.

In the context of this patent the expression “target gene or locus”, “target material” or “target DNA” defines a specific region in the genetic material of the cell intended for modification. The nucleic acid molecule subject of the invention has to be adapted based on the sequence of the target gene or locus. The design requires to provide the molecule with two H-regions homologous to the target material, as well as to adapt the sequence encoding the nuclease recognition and binding site present in the R-region to recognize and cut a sequence in the target DNA.

The molecule subject of this invention and the related gene editing method may be used advantageously to edit the two alleles of a target gene or locus in the cell genetic material, being able to change ad lib the sequence encoded by that gen without leaving a “genetic scar”. In the context of this patent, by “genetic scar” is understood any residual sequence which unintentionally has been inserted definitively in the genetic material of the modified cell.

Another aspect of the invention consists in the use of the molecule as therapeutic composition and its medical use as drug or treatment of a large amount of genetic disorders caused by an anomalous codification in the genome, such as a way of example and without limitation: sialidosis, galactosialidosis, alpha-mannosidosis, beta mannosidosis, aspartylglucosaminuria, fucosidosis, Schindler disease, metachromatic leukodystrophy, multiple sulfatase deficiency, globoid cell leukodystrophy (or Krabbe disease), glycogen storage disease type II (or Pompe disease), Farber disease (or Farber's lipogranulomatosis), lysosomal acid lipase deficiency (or Wolman's disease), cholesteryl ester storage disease, pycnodysostosis, ceroid lipofuscinosis types 6 and 8, cystinosis, Salla disease, mucolipidosis types III and IV, Danon disease, Chediak-Higashi syndrome, Griscelli syndrome types 1, 2 and 3, Hermansky-Pudlak syndrome type 2, X linked juvenile retinoschisis, Stargardt disease, choroideremia, Retinitis Pigmentosa types 1 to 56, achondroplasia, achromatopsia, acid maltase deficiency, adenosine deaminase deficiency, adrenoleukodystrophy, Aicardi syndrome, alpha-1 antitrypsin deficiency, alpha thalassemia, androgen insensitivity syndrome, Apert syndrome, arrhythmogenic right ventricular dysplasia, ataxia telangiectasia, Barth syndrome, beta-thalassemia, Canavan disease, blue rubber bleb nevus syndrome (or Bean syndrome), chronic granulomatous disease, Cri du chat syndrome, cystic fibrosis, adiposis dolorosa (or Dercum's disease), ectodermal dysplasia, Fanconi anemia, fibrodysplasia ossificans progressiva, fragile X syndrome, galactosemia, Gaucher disease, gangliosidosis, hemochromatosis, hemoglobinopathy by hemoglobin C (HbC), hemophilia, Huntington's disease, Hurler syndrome, hypophosphatasia, Klinefelter syndrome, Langer-Giedion syndrome, leukocyte adhesion deficiency, leukodystrophy, long QT syndrome, Marfan syndrome, Moebius syndrome, mucopolysaccharidosis, nail-patella syndrome, neurofibromatosis, nephrogenic diabetes insipidus, osteogenesis imperfecta, Niemann-Pick diseases, porphyria, Prader-Will syndrome, progeria, Proteus syndrome, retinoblastoma, Rubinstein-Taybi syndrome, Rett syndrome, Sanfilippo syndrome, severe combined immunodeficiency, Shwachman-Diamond syndrome, sickle-cell disease, Smith Magenis Syndrome, Stickler syndrome, Tay-Sachs disease, thrombocytopenia-absent radius syndrome, Down syndrome, Treacher Collins syndrome, trisomy, tuberous sclerosis, X-linked lymphoproliferative syndrome, Turner syndrome, urea cycle disorder, von Hippel-Lindau disease, Waardenburg syndrome, Williams Syndrome, Wilson disease and Wiskott-Aldrich syndrome.

The use of the nucleic acid molecule as therapeutic composition and its usage as drug for the treatment or prevention of a large variety of genetic disorders, supposes the administration of a therapeutic amount of the molecules subject of the invention and/or an amount of modified cells (by means of the molecule subject of the invention) in an experimental model, organism or ill subject.

In a particular embodiment, the therapeutic composition will be used to treat the acquired immune deficiency syndrome (AIDS) caused by the human immunodeficiency virus (HIV), through the disruption or modification by gene editing by means of the nucleic acid molecule subject of the invention of membrane receptors used by viruses and bacteria, turning them useless for the pathogen to be internalized or to interact with the cells.

The expression “therapeutic amount” in the context of this invention is referred to the amount of the therapeutic composition of the molecule which quantity, after the administration, is enough to prevent or treat one or more symptoms of the disease, being therefore used as a medicine. Another aspect of the invention has a preferred application in a treatment and/or prevention of the acquired immune deficiency syndrome (AIDS) caused by the human immunodeficiency virus (HIV): In a particular embodiment of the nucleic acid molecule subject of the invention, the selected target gene is the CCR5 gene (C-C chemokine receptor type 5) which encodes for a membrane coreceptor used by the R5-tropic HIV to be internalized and to infect T-cells and reservoir cells.

The method and molecule subject of the invention adapted to the CCR5 gene, applied to T-cells and/or their precursors as a way of example and without limitation, hematopoietic stem cells (HSC), generate ultimately modified T-cells and/or T-cell precursors modified with the CCR5 gene edited in the two alleles, in a way in which the receptor, once it is expressed, doesn't allow the binding of the HIV virus through the viral proteins gp120 and gp41.

An example of gene editing in the two alleles of the CCR5 allele by means of this method is to generate the allelic variant CCR5-Δ32, which is HIV resistant. The T-cells and/or T-cell precursors generated by this method will have resistance to the HIV internalization. Once they are introduced in a patient, they will remove the cells infected by HIV (native cells, reservoir cells and others) providing a cure for the AIDS.

To obtain a permanent protection against the HIV, HSC cells are extracted from the patient and then transplanted back after the editing of both CCR5 alleles by means of the nucleic acid molecule subject of the invention.

### EXAMPLES

### Example 1: Adjustment of the Nucleic Acid Molecule for the Biallelic Editing of the CCR5 Gene in Homo sapiens According to the Method in this Invention

The adjustment of the nucleic acid molecule comprises to provide two homology regions, defined in the context of this patent as H-regions, such as, in this case SEQ ID #1 for the first H-region (corresponding to the 5′ homology branch) and SEQ ID #2 for the second H-region (3′ homology branch) following the 5′ to 3′ direction of transcription.
In this example the nucleic acid molecule doesn't carry any E-region, but the 5′ homology branch presents a 32 bp deletion with regards to the sequence in the cell genetic material for the CCR5 gene. This deletion is characteristic of the CCR5-Δ32 allele, which confers resistance to the HIV internalization.
Another aspect of this adjustment is the modification of the guide RNA (gRNA) used by the ribonuclease protein CRISPR/Cas9 to recognize the cleavage site of the target gene or locus. In this case the gRNA is designed as in the SEQ ID #3, but it could be used also as example and without limitation, another sequence annex to a PAM motif, close to the cleavage site as for example SEQ ID #4. The design of the DNA is known by the specialist scientist.
The TRT region is placed between the two H-regions encoding the beginning and the end of the piggyBac transposon. Within the R-region is present the N-sequence which encodes for the protein CRISPR/Cas9 and the gRNA designed for the sequence SEQ ID #3 or SEQ ID #4 and the other sequences according to the patent claims.
In this example, after the scission of the transposable element, both alleles of the CCR5 gene are edited to CCR5-Δ32 without leaving any genetic scar.

### Example 2: Alternative Adjustment of the Nucleic Acid Molecule for the Biallelic Editing of the CCR5 Gene in Homo sapiens According to the Method Described in this Invention

The adjustment of the nucleic acid molecule comprises to provide two homology regions, defined in the context of this patent as H-regions, such as, in this case SEQ ID #5 for the first H-region (corresponding to the 5′ homology branch) and SEQ ID #2 for the second H-region (3′ homology branch) according to the 5′ to 3′ direction of transcription.
In this example the molecule carries a E-region, located between position 963 and 980 within the first H-region in the molecule according to the 5′ to 3′ direction of transcription. This E-region is needed to modify the recognition site and DNA binding site of the TALEN nuclease. In this example the E-region carries only silent mutations which don't alter the information encoded in the DNA for the amino acid sequence of the protein. Likewise this first H-region carries a 32 bp deletion with regards to the CCR5 gene sequence present in the genetic material of the cell. This deletion is characteristic of the CCR5-Δ32 allele, which confers resistance to the HIV internalization.
Another aspect of this adjustment is the modification of the recognition sequences of the TALEN nucleases in the CCR5 target gene. In this case the two complementary TALEN nucleases are designed to recognize the sequence SEQ ID #6 and SEQ ID #7. The recognition of these sequences by means of the TALEN nucleases is achieved by modifying the RVD sequence of the nucleases, know by the specialist scientist.
The TRT regions is placed between the two H-regions and encodes the beginning and the end of the piggyBac transposon. Within the R-region is present the N-sequence which encodes for the two TALEN nucleases and the other sequences according to the patent claims.
In this example, after the scission of the transposable element, the transcription and traduction of the two modified alleles will result in the expression of the CCR5 variant protein CCR5-Δ32, however, in the sequence of both alleles will be present the silent mutations of the E-region (bases #963, 965, 966, 968, 971, 974, 977, 980 from SEQ ID #5).

The names H, T, R and E-regions and M, N, S and P-sequences used in this patent are merely explanatory and their purpose is to define the structure and composition of the nucleic acid molecule subject of the invention. The name of the defined sequences is not relevant nor limiting of the invention.

### DESCRIPTION OF THE FIGURES

For a better understanding of the invention several figures are attached to this document with the purpose of illustrating some of the explained concepts, but not limiting the extent of the invention.

FIG. 1 shows a diagram of the structure of the nucleic acid molecule subject of this invention. The order in which H, T and R regions are detailed follows the transcription direction 5′ to 3′. In this diagram the E region is not detailed, neither the composition of sequences of the R-region.

FIG. 2 shows a diagram of the structure of the nucleic acid molecule subject of this invention, detailing examples for the possible location of punctual modifications to be incorporated to the cellular DNA (E region). a) Particular embodiment of the nucleic acid molecule that contains a deleted sequence between H regions (used to delete in the edition procedure a sequence from the edited gen of interest or locus). b) Particular embodiment of the nucleic acid molecule that contains an E region defined by one to several nucleobase modifications. c) Particular embodiment of the nucleic acid molecule that contains an E region in the terminal portion of the 5′ H homology branch. d) Particular embodiment of the nucleic acid molecule that contains an E region in the initial portion of the 3′ H homology branch.

FIG. 3 shows a diagram of a particular embodiment of the nucleic acid molecule subject of this invention (3a), designed to remove a specific sequence in the target gene or locus from the cellular DNA, and shows also the gene edition process that would be executed following the application of the invention. a) DNA alleles from the gene of interest or locus to be edited and particular embodiment of the nucleic acid molecule. The cut that will be caused by the nuclease activity expressed by the N region is shown. The recombination loops formed by the HDR mechanism between the particular embodiment of the nucleic acid molecule and one of the present alleles is also indicated. b) Non edited allele and edited one by ‘HTNMSPTH’ integration depicting a cut caused by the nuclease activity from the product expressed by the integrated N region and the recombination loops formed by the HDR mechanism between the edited allele and the wild type one. c) Bi-allelic gene edition performed, both alleles harbor a ‘HTNMSPTH’ integration. d) Excision of the ‘TNMSPT’ region from both alleles due to transposon activation, leaving only the H regions that contains the edited sequence (may be point mutations, deletions or one to several nucleobase modifications depending of the particular embodiment of the nucleic acid molecule used to carry the bi-allelic gene edition)

FIG. 4 shows a diagram of the structure of the nucleic acid molecule subject of this invention detailing sequences N, M, S and P, which resides within the R region. The order or disposition of these sequences is not relevant in the transcription direction 5′ to 3′. In this diagram the E region is not detailed.

FIG. 5 shows a diagram of a particular embodiment of the nucleic acid molecule subject of this invention, designed to insert a specific sequence ‘E’ into the target gene or locus present in the cell. The figure shows also the gene edition process carried out according to the embodiment of the invention (5a, b, c, d, and e). Region E location can be in either one of the two ‘H’ regions or both, being that location irrelevant for the gene edition process as long as they are within the Hollyday junction zone. a) Cleavage by the nuclease encoded in ‘N’. b) Integration of the ‘HETNMSPTH’ region. c) Cleavage of the remaining allele; 5d) Copy of the recombined allele into the second allele cut by HDR. e) ‘TNMSPT’ transposon excision in both alleles leaving only the desired specific sequence ‘E’ at the desired position of the gene or locus to be modified.

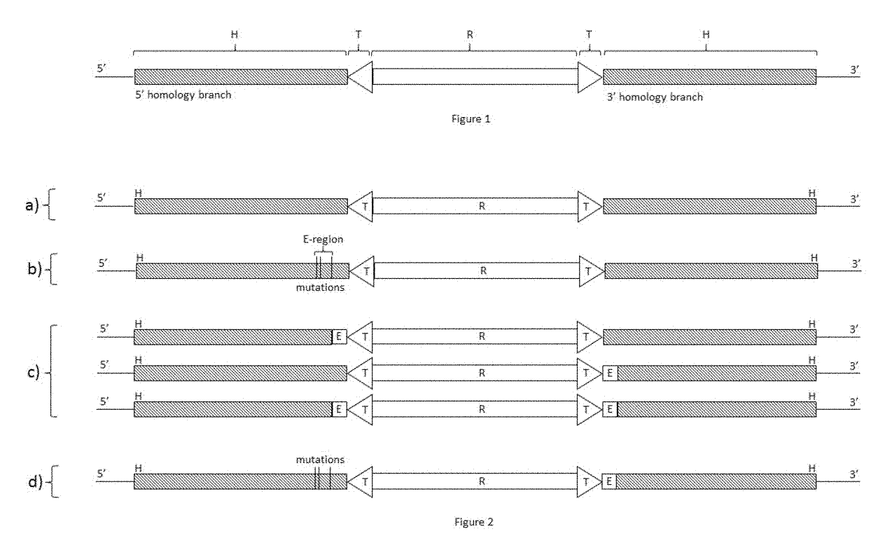

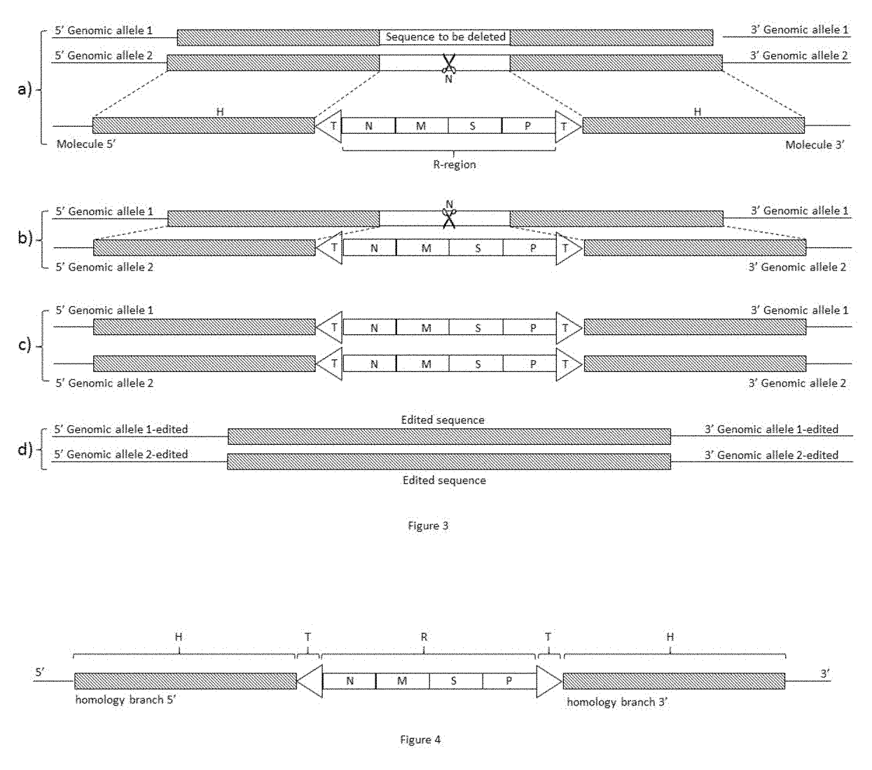

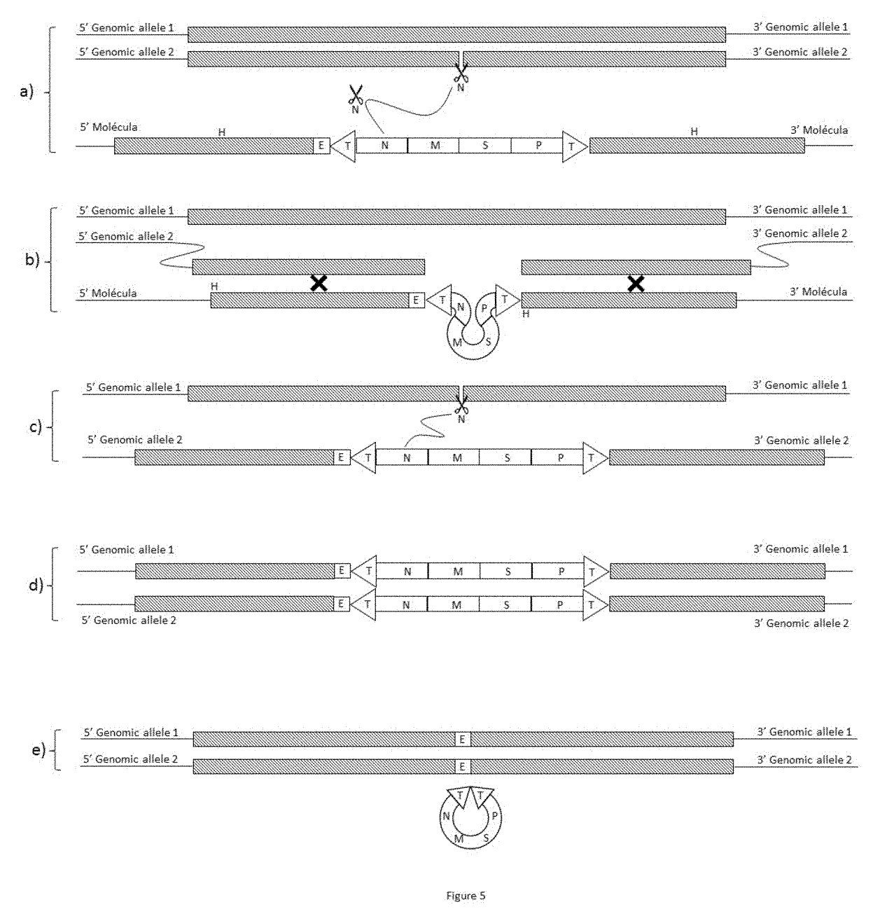

### REFERENCES

1. Moore, J. K. and J. E. Haber, Cell cycle and genetic requirements of two pathways of nonhomologous end-joining repair of double-strand breaks in Saccharomyces cerevisiae. Mol Cell Biol, 1996. 16(5): p. 2164-73.
2. Burt, A. and V. Koufopanou, Homing endonuclease genes: the rise and fall and rise again of a selfish element. Curr Opin Genet Dev, 2004. 14(6): p. 609-15.

1. Nucleic acid molecule composed by: Two H-regions homologous to the gene or locus to edit, two T-regions which contain a transposable element (as a way of example and without limitation: the piggyback transposon, the sleeping Beauty transposon or any other transposon), one R-region which carries the coding sequences for proteins needed to carry out the method of biallelic gene editing, and additionally one or more genetic modifications to be incorporated to the cell genetic material, called E-regions.

2. Nucleic acid molecule according to the claim #1 in which the E-regions are independent of the H-regions or being present within the H-regions in the way of at least one mutation with regards to the homology region.

3. Nucleic acid molecule according to the claim #1, in which its constitutive elements are arranged in the 5′ to 3′ direction of transcription in the following order: either HTRTEH or HETRTH.

4. Nucleic acid molecule according to the claim #1, in which its constitutive elements are arranged in the 5′ to 3′ direction of transcription in the following order: HETRTEH.

5. Nucleic acid molecule according to the claim #1, in which its constitutive elements are arranged in the 5′ to 3′ direction of transcription in the following order: HTRTH, being the genetic modifications that have to be incorporated to the cell genome in at least one of the H-regions.

6. Nucleic acid molecule according to the claim #1, in which its constitutive elements are arranged in the 5′ to 3′ direction of transcription in the following order: HTRTH, and without genetic modifications to be incorporated to the cell genome. This molecule may be used to remove a specific sequence (at least 1 bp) from the target gene or locus present in the cell genetic material. After the gene editing is executed, a segment of the target gene or locus is removed due to a separation in the codification between the two H-regions present in the nucleic acid molecule subject of the invention. For further reference about the method, the two specific examples described in the patent use this methodology (see section “Detailed description of the invention”, lines 528 to 583, pages 18 and 19).

7. Nucleic acid molecule according to the claim #6, in which the R-region encodes for the following proteins: at least a nuclease protein (N) and at least a selection protein (S).

8. Nucleic acid molecule according to the claim #7 in in which the coding sequence for the proteins N-S is arranged in the R-region as either separated genes, or by means of polycistronic genes, or by a mix of separated and polycistronic genes together. The order of the genes in the 5′ to 3′ direction of transcription (N-S or S-N) is irrelevant for the method described in this patent.

9. Nucleic acid molecule according to the claims #7, in which the R-region further encodes one or more marker proteins (M) and/or one or more cell proliferation proteins (P). These additional sequences are not essential for the correct performance of the method but assist in related tasks.

10. Nucleic acid molecule according to the claim #9 in which the coding sequence for the proteins NSMP is arranged in the R-region as separated genes, or by means of polycistronic genes, or by a mix of separated and polycistronic genes together. The order of the genes in the 5′ to 3′ direction of transcription (N-S or S-N) is irrelevant for the method described in this patent.

11. Nucleic acid molecule according to the claims #1 in which the R-region consists only of coding sequences for nuclease proteins (N), coding sequences for marker proteins (M) and coding sequences for cell proliferation proteins (P).

12. Nucleic acid molecule according to the claim #7, which encodes nuclease proteins as a way of example and without limitation: homing endonucleases (HEs), zinc finger nucleases (ZFN), TALEN nucleases (from transcription activator-like effector nucleases) or RNA-dependent DNA endonucleases from the CRISP/Cas9 system (from clustered regulatory interspaced short palindromic repeats) and their corresponding gRNA (guide RNA). These nucleases may be of the previously described types but they are not limited to them.

13. Nucleic acid molecule according to the claims #1, in which the H-regions are at the ends of the nucleic acid molecule flanking all the construction. These H-regions show sequence analogy with the region of the cell genetic material where the nuclease proteins (N) perform the cleavage.

14. Nucleic acid molecule according to the claim #1, in which the H-regions comprise at least one mutation (a change in the sequence) for, as a way of example and without limitation: modifying the target gene correcting its function or preventing it, and/or generating restriction sites or removing them, and/or generating primer binding sites or removing them, and/or generating nuclease recognition and binding sites or removing them. This mutation differentiates the modified gene from the native one without altering its codification.

15. Nucleic acid molecule according to the claim #1, in which the H-region have a length between 50 bp and 10 kbp, being their preferred length 900 bp.

16. Nucleic acid molecule according to the claim #7, in which the S-sequence residing within the R-region, encodes at least for one resistance and selection protein (S) which is able to generate a positive selection event (cell survival) against the presence of a selection agent such as for example and without limitation, antibiotics or other compounds toxic for the cell.

17. Nucleic acid molecule according to the claim #9, in which the M-sequence residing within the R-region, encodes for one or more marker proteins (M). These proteins may be, as a way of example and without limitation: fluorescent protein markers (green fluorescent protein (GFP), Turbo GFP, copGFP, tdTomato, infrared fluorescent protein (IRFP), mEmerald, Venus, super yellow fluorescent protein 2 (SYFP2), DsRed, enhanced blue fluorescent protein (EBFP), enhanced yellow fluorescent protein (EYFP), Cerulean, enhanced cyan fluorescent protein (ECFP) and others), cell surface marker proteins (leukocyte differentiation markers and clusters of differentiation (CD)) or any other membrane protein that can be used to detect and isolate the cell that is expressing it.

18. Nucleic acid molecule according to the claim #9, in which the P-sequence residing within the R-region, encodes for one or more cell proliferation proteins (P), understanding as “cell proliferation protein” any protein expressed inside the cell which stimulates its proliferation and divisions; or proteins that inhibits apoptotic pathways, immortalization proteins, or any protein which activity or product confers an advantage by selective growth in the presence of its substrate as well in its absence. Some example of proliferation proteins are, without limitation, inhibitor of apoptosis proteins (IAP's), caspase activation pathway inhibitors (crmA, p35, Bcl-2, etc.) and immortalization proteins (EBNA-LP, hTERT, H2RSP, etc.).

19. Nucleic acid molecule according to the claim #9, in which the P-sequence residing within the R-region, encodes for one or more “interfering RNA”, as a way of example and without limitation small interfering RNA (siRNA), microRNA (miRNA) and PIWI-interacting RNA (piRNA), which activity triggers an event which stimulates cell proliferation and/or cell division and/or apoptosis pathway inhibition and/or cell immortalization.

20. Nucleic acid molecule according to the claim #1, in which the nucleic acid molecule consists in at least one region containing the genetic modifications that are intended to be introduced permanently in the cell genetic material (E-region). Such E-region may be introduced within the H-regions in the way of at least one punctual modification of the sequence or being between the H-T regions and/or T-H regions encoding a protein, part of a gene, an intron, an exon or a whole gene.

21. Nucleic acid molecule according to the claim #1, in which said nucleic acid is either a molecule of deoxyribonucleic acid and/or one or several ribonucleic acid molecules, being either a double strand or a single strand molecule, either in circular or linear form.

22. Nucleic acid molecule according to the claim #1, in which said nucleic acid contains polycistronic and/or monocistronic genes.

23. Method to modify the cell genetic material such that the modification occurs in both alleles of the target gene or locus, and is identical in both of them, comprising following stages: i) provide and introduce in the said nucleic acid molecule; ii) select the cells that have integrated the said molecule in both alleles of the target gene or locus; iii) trigger the scission of part of the said integrated molecule such that only the desired modifications (sequence substitutions, additions or deletions) remain in both alleles of the cell genetic material of the modified cell; iv) select the cells which have the desired modifications in both alleles.

24. Method according to the claim #23 in which the cells, having their genetic material modified, are identified and selected by means of the detection of fluorescent protein markers encoded in the introduced nucleic acid molecule.

25. Method according to the claim #23 in which the cells, having their genetic material modified, are identified and selected by means of the detection of surface protein markers encoded in the introduced nucleic acid molecule.

26. Method according to the claim #23 in which the cells, having their genetic material modified, are identified and selected by means of the activity of the resistance and selection proteins encoded in the introduced nucleic acid molecule.

27. Method according to the claim #23 in which the cells, having their genetic material modified, suffer the removal of the transposable sequence (TRT) by means of the activation of the related recombinase protein.

28. Method according to the claim #27 in which the cells, having their genetic material modified, are selected based on the absence of function of the negative selection proteins encoded in the nucleic acid molecule and/or the absence of fluorescence from the fluorescent protein markers and/or the absence of signal from other surface marker proteins.

29. Method according to the claim #23 in which the genetic material is introduced in the cell by means of a viral vector and/or a non-viral vector system.

30. Method according to the claim #23 in which the target gene is the CCR5 gene (C-C chemokine receptor type 5) which encodes for a membrane coreceptor used by the R5-tropic HIV to be internalized in T-cells.

31. Method according to the claim #30 in which the modification of the genetic material generates a T-cell strain with the CCR5 membrane coreceptor gene edited in both alleles in such a way that its expression makes not possible its use for the R5-tropic HIV to enter and infect those T-cells.

32. Method according to the claim #30 in which the modification of the genetic material generates a T-cell precursors strain, as a way of example and without limitation, hematopoietic stem cells (HSC) or induced pluripotent stem cells (iPS) with the CCR5 membrane coreceptor gene edited in both alleles in such a way that its expression makes not possible its use for the R5-tropic HIV to enter and infect those T-cells.

33. Therapeutic composition comprising at least one nucleic acid molecule according to the claim #1.

34. Therapeutic composition according to the claim #33 for the treatment of hereditary diseases.

35. Therapeutic composition according to the claim #33 for the treatment of the acquired immune deficiency syndrome (AIDS) caused by the human immunodeficiency virus (HIV).


SEQUENCE LISTING (7 sequences)

SEQ ID NO: 1 - DNA, 996 bp, Homo sapiens
tccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccataca

SEQ ID NO: 2 - DNA, 1060 bp, Homo sapiens
agatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagc

SEQ ID NO: 3 - DNA, 23 bp, Homo sapiens
catacagtcagtatcaattctgg

SEQ ID NO: 4 - DNA, 23 bp, Homo sapiens
aaagatagtcatcttggggctgg

SEQ ID NO: 5 - DNA, 996 bp, Homo sapiens
tccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtttacactatacatgtagttctcattttccataca

SEQ ID NO: 6 - DNA, 17 bp, Homo sapiens
tcattacacctgcagct

SEQ ID NO: 7 - DNA, 17 bp, Homo sapiens
cttccagaattgatact